(19) United States
(12) Patent Application Publication
(10) Pub. No.: US 2011/0208204 A1
(43) Pub. Date: Aug. 25, 2011

(54) ARTHROSCOPIC ACCESS TO THE INTERIOR OF A HIP JOINT

(76) Inventors: Hal David Martin, Oklahoma City, OK (US); James Flom, San Carlos, CA (US); Julian Nikolchev, Portola Valley, CA (US)

(21) Appl. No.: 13/020,680

(22) Filed: Feb. 3, 2011

Related U.S. Application Data
(63) Continuation-in-part of application No. 12/631,514, filed on Dec. 4, 2009.
Application No. 12/631,514 is a continuation-in-part of application No. 12/726,268, filed on Mar. 17, 2010.
(60) Provisional application No. 61/301,005, filed on Feb. 3, 2010.

(51) Int. Cl. A61B 17/00

(52) U.S. Cl. 606/108

(57) ABSTRACT

A method for arthroscopically accessing a region of a joint, wherein the joint has a capsule disposed intermediate at least one layer of outer tissue and the joint, the method comprising:
arthroscopically positioning visualization apparatus adjacent to an exterior surface of the capsule; and
while visualizing the exterior surface of the capsule, arthroscopically forming an opening through the capsule.

### REFERENCE TO PENDING PRIOR PATENT APPLICATIONS

This patent application:

(i) is a continuation-in-part of pending prior U.S. patent application Ser. No. 12/631,514, filed Dec. 4, 2009 by James Flom et al. for METHOD AND APPARATUS FOR ACCESSING THE INTERIOR OF A HIP JOINT, INCLUDING THE PROVISION AND USE OF A NOVEL TELESCOPING ACCESS CANNULA AND A NOVEL TELESCOPING OBTURATOR (Attorney's Docket No. FIAN-3143);

(ii) is a continuation-in-part of pending prior U.S. patent application Ser. No. 12/726,268, filed Mar. 17, 2010 by Julian Nikolchev et al. for METHOD AND APPARATUS FOR DISTRACTING A JOINT, INCLUDING THE PROVISION AND USE OF A NOVEL JOINT-SPACING BALLOON CATHETER AND A NOVEL INFLATABLE PERINEAL POST (Attorney's Docket No. FIAN-28424953); and

(iii) claims benefit of pending prior U.S. Provisional Patent Application Ser. No. 61/301,005, filed Feb. 3, 2010 by Hal David Martin for ARTHROSCOPIC ACCESS TO THE INTERIOR OF THE HIP JOINT (Attorney's Docket No. PROV).

The three (3) above-identified patent applications are hereby incorporated herein by reference.

### FIELD OF THE INVENTION

This invention relates to surgical methods and apparatus in general, and more particularly to surgical methods and apparatus for treating a hip joint.

### BACKGROUND OF THE INVENTION

Successful hip arthroscopy generally requires safe and effective access to the interior of the hip joint.

The current technique for arthroscopically accessing the interior of a hip joint generally comprises the following steps. First, the patient's leg is typically placed under traction so as to dislocate the femoral head from the acetabular socket. This action creates a gap, or opening, between the femoral head and the acetabular socket, thereby allowing the interior surfaces of the joint to be accessed. Under fluoroscopic guidance, and looking now at FIG. 1, the surgeon then advances a hollow needle 5 (temporarily filled with a stylet 10) from the surface of the skin 15 down into this gap, so that that the tip of needle 5 resides in the interior of the joint. As the tip of needle 5 passes from the surface of the skin 15 down into the gap between the femoral head and the acetabular cup, the needle (with stylet) must pass through the intervening tissue, which includes the outer tissue 20 (skin, muscle, etc.) and the tough band of tissue (i.e., the capsule 25) which surrounds the joint. After needle 5 (with stylet 10) has been advanced into the interior of the joint, stylet 10 is removed from needle 5, as seen in FIG. 2. Next, a guidewire 30 is advanced through needle 5 and into the interior of the joint (FIG. 3). Needle 5 is then removed (FIG. 4), leaving guidewire 30 extending from the surface of the skin 15 down into the interior of the joint. Next, a skin incision may or may not be made about guidewire 30. Then an access cannula 35 is advanced over guidewire 30 and down into the interior of the joint (FIG. 5). Guidewire 30 is then removed (FIG. 6).

The foregoing procedure is then typically repeated so as to deploy additional access cannulas 35 into the joint (FIG. 7.)

Once the desired access cannulas 35 have been installed in the anatomy, an arthroscope 40 (FIG. 8) is advanced through one of the access cannulas 35 so as to visualize the interior of the joint. Other arthroscopic instrumentation (e.g., a burr 45, as shown in FIG. 9) may then be advanced down other access cannulas so as to treat the interior of the joint.

Unfortunately, it is not uncommon for needle 5 to cause iatrogenic damage to tissue structures as the needle is advanced into the interior of the joint. For example, needle 5 may accidentally penetrate and damage the labrum, which is a soft tissue structure located on the rim of the acetabulum. Or, the needle may scuff or gouge cartilage on the femoral head. Or the needle may damage cartilage on the inner surface of the acetabular cup. In many cases, this iatrogenic damage is caused by “needle plunge”, which often occurs as the needle is forced through the tough capsule which surrounds the joint. More particularly, the surgeon typically needs to apply substantial force to the proximal end of the needle in order to force the distal end of the needle through the tough capsule, but it can then be very difficult for the surgeon to stop the needle from plunging forward into underlying anatomical structures when the needle finally breaks through the tough capsule. When this occurs, the underlying anatomical structures (e.g., the labrum, the head of the femur, the acetabular cup, etc.) can be damaged by such a needle plunge.

Thus there is the need for a safer approach for arthoscopically accessing the interior of a hip joint.

### SUMMARY OF THE INVENTION

The present invention provides a safer approach for arthroscopically accessing the interior of a hip joint.

In one preferred form of the present invention, there is provided a method for arthroscopically accessing a region of a joint, wherein the joint has a capsule disposed intermediate at least one layer of outer tissue and the joint, the method comprising:

arthroscopically positioning visualization apparatus adjacent to an exterior surface of the capsule; and

while visualizing the exterior surface of the capsule, arthroscopically forming an opening through the capsule.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1-9 are schematic views showing a conventional approach for achieving arthroscopic access to the interior of a hip joint;

FIGS. 10-24 are schematic views showing a new approach for achieving arthroscopic access to the interior of a hip joint; and

FIGS. 25-29 are schematic views showing another new approach for achieving arthroscopic access to the interior of a hip joint.

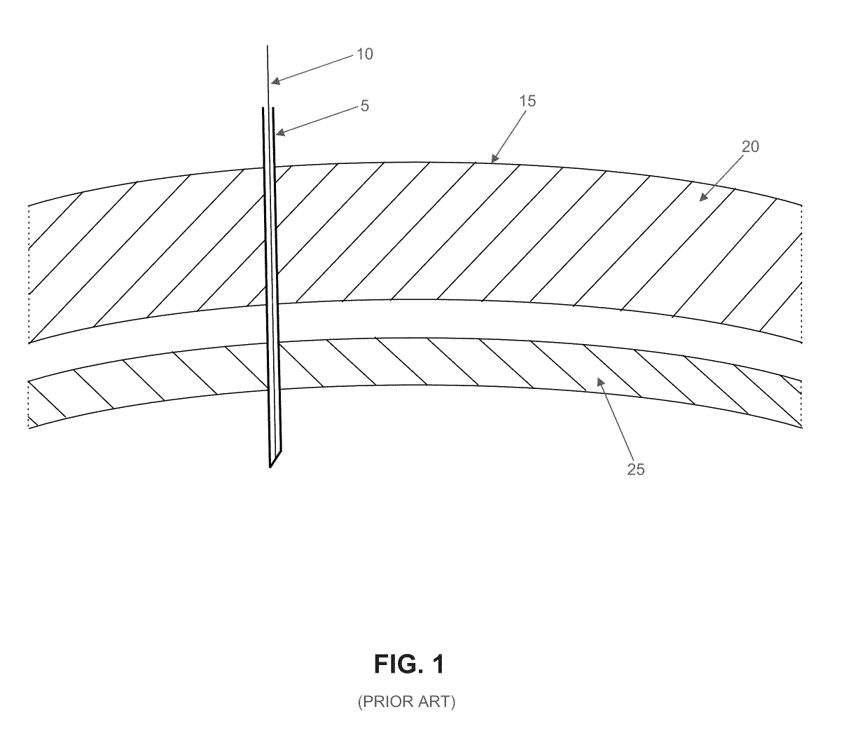

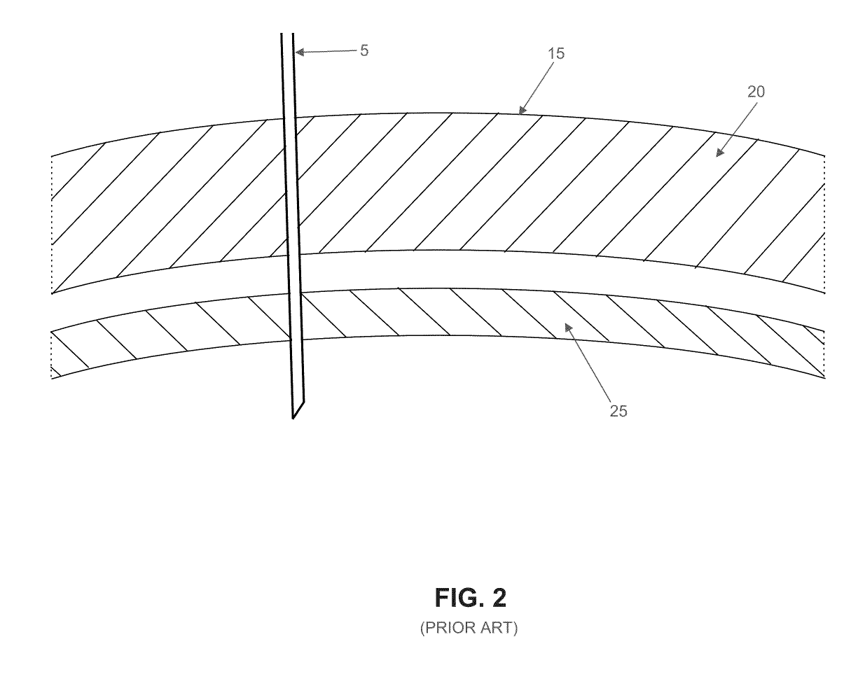

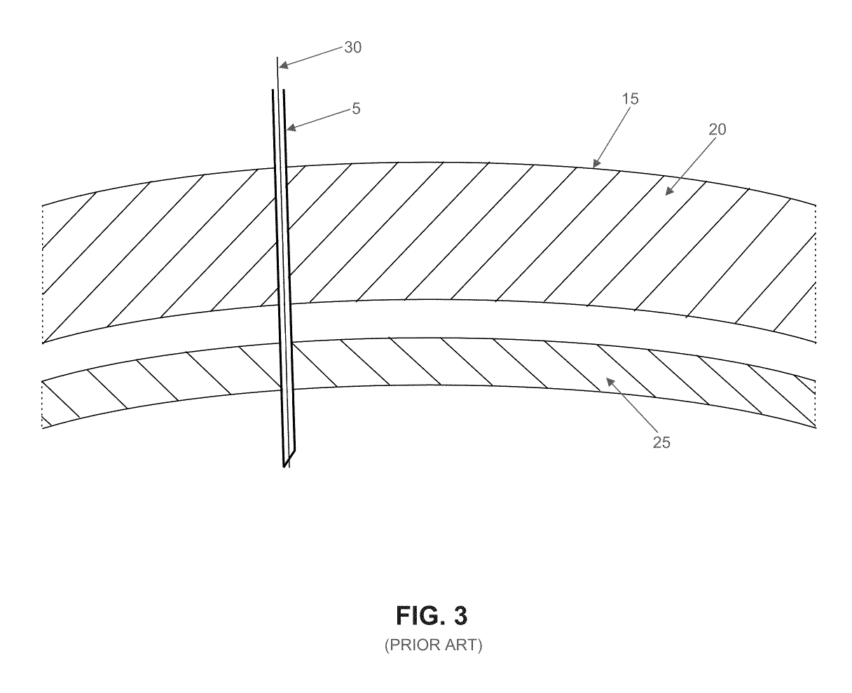

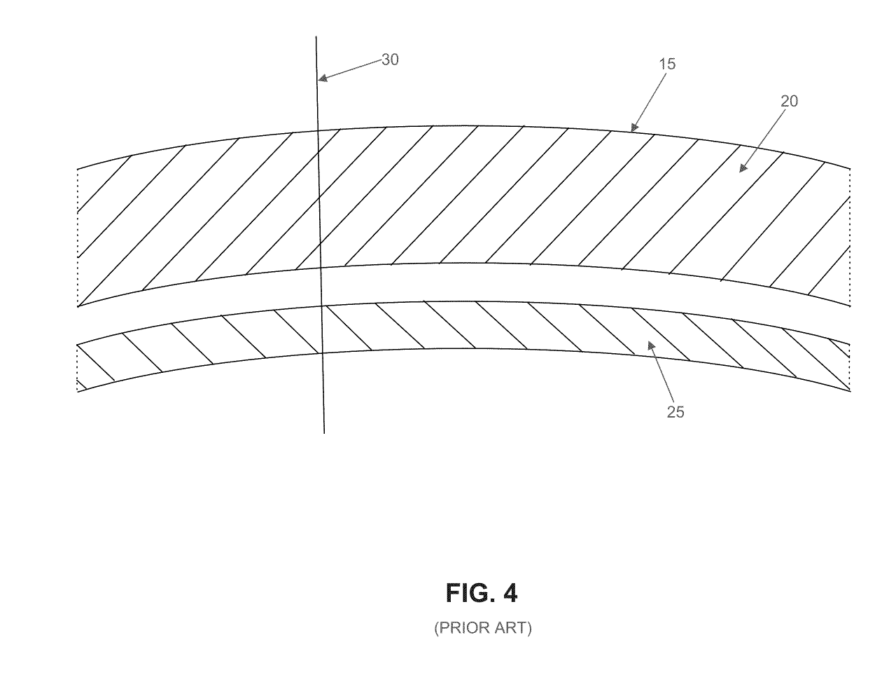

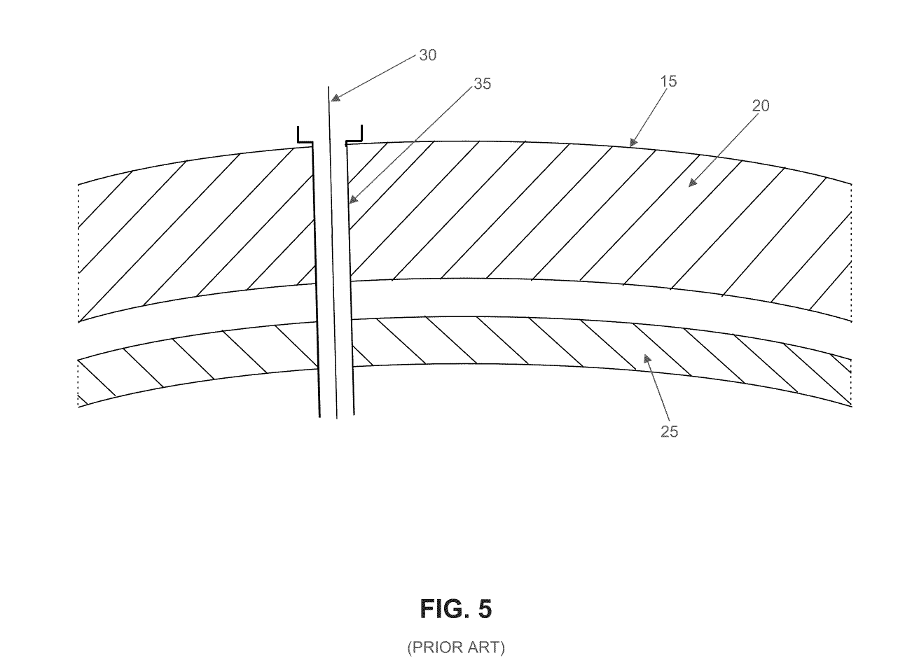

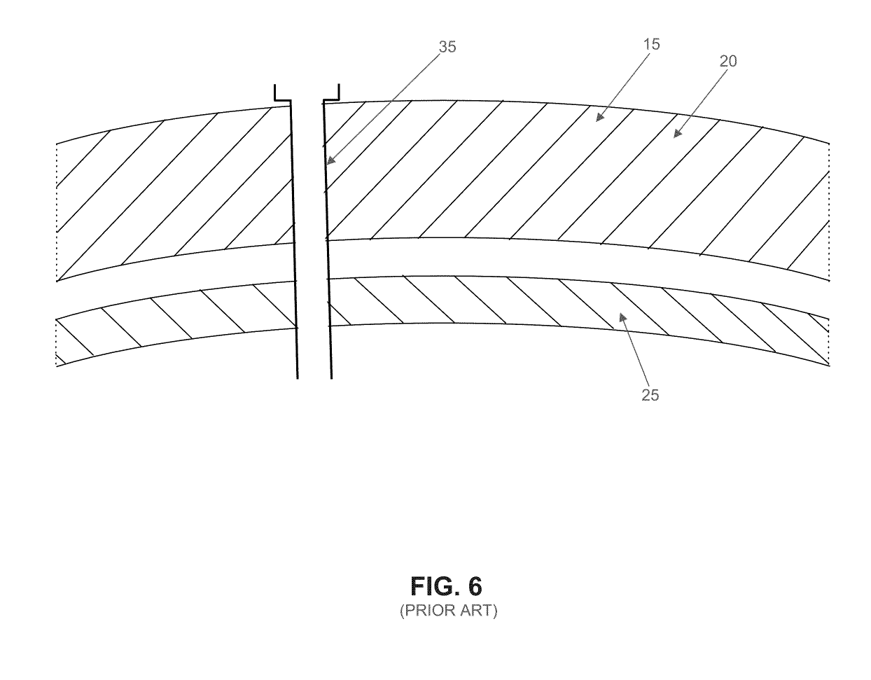

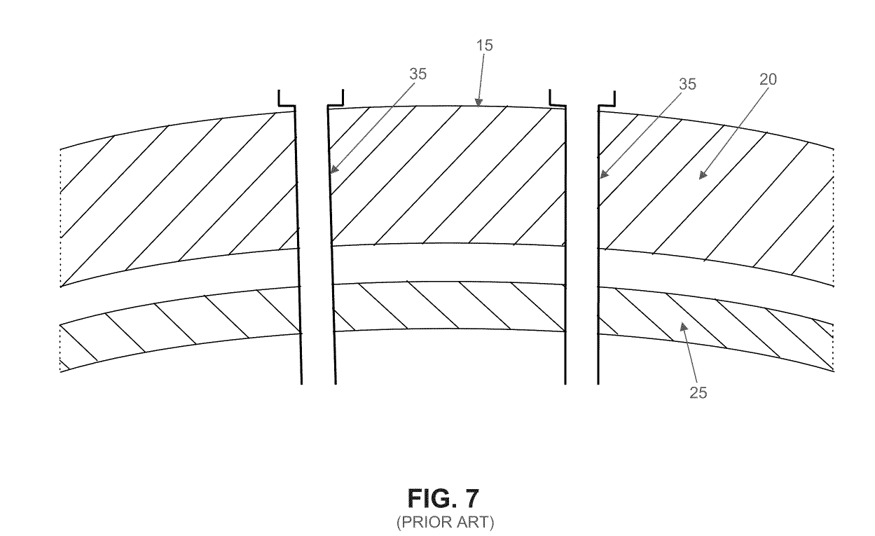

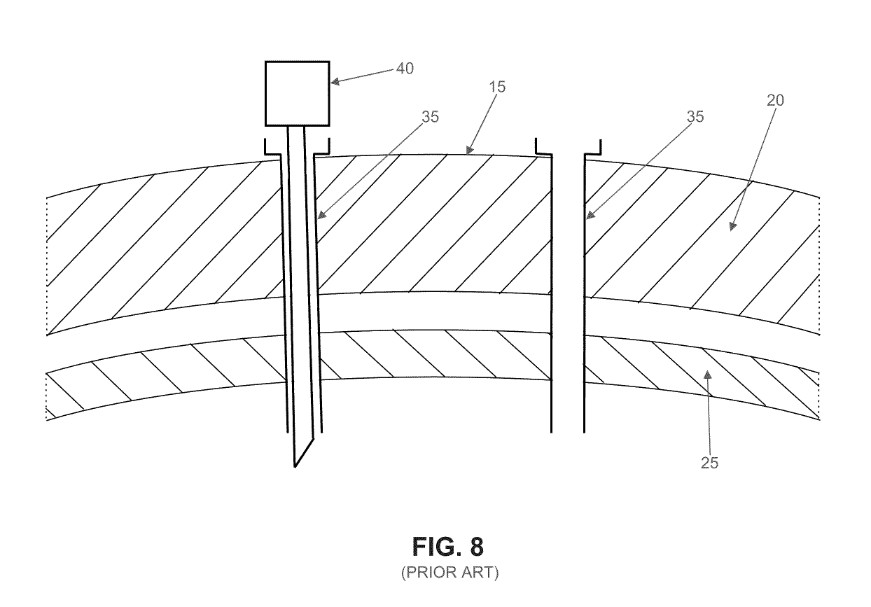

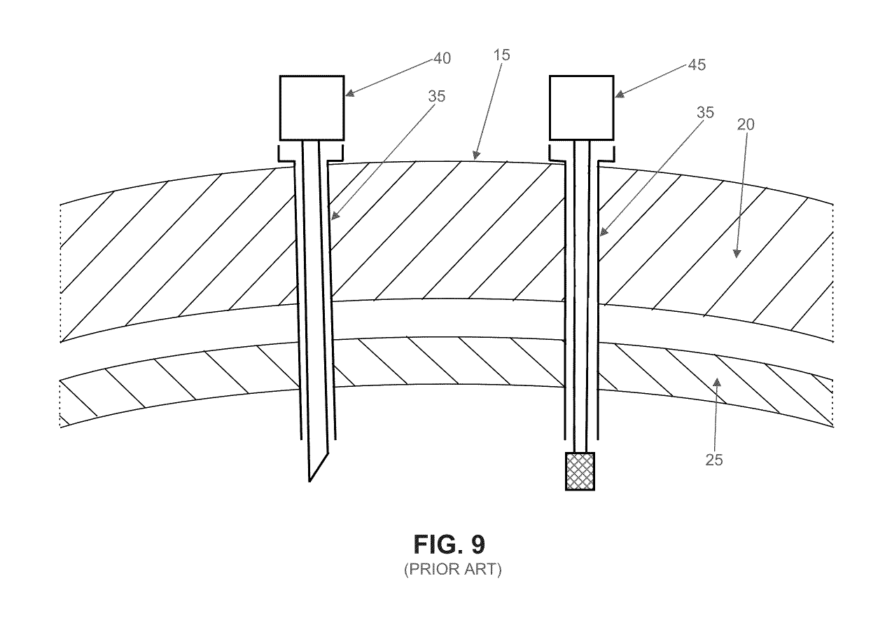

### DETAILED DESCRIPTION OF THE PREFERRED EMBODIMENTS

The present invention provides a safer approach for arthroscopically accessing the interior of a hip joint.

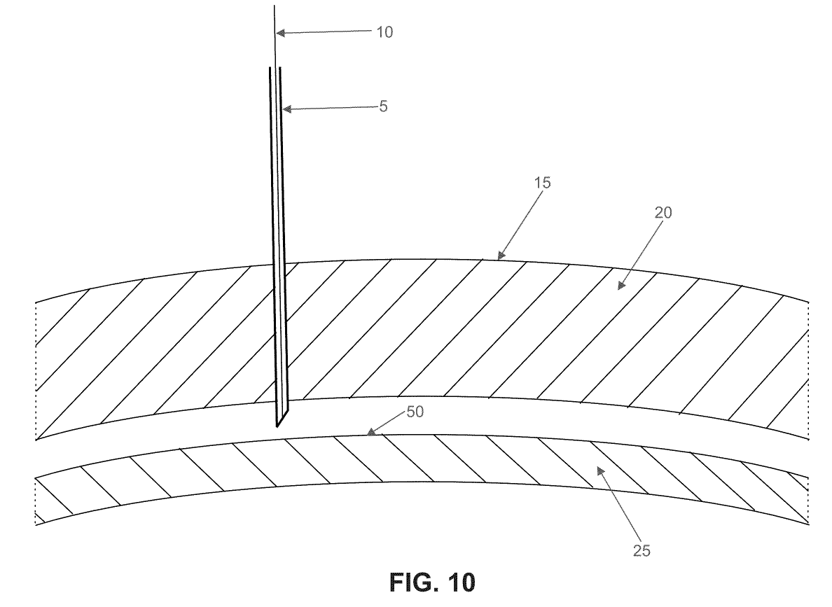

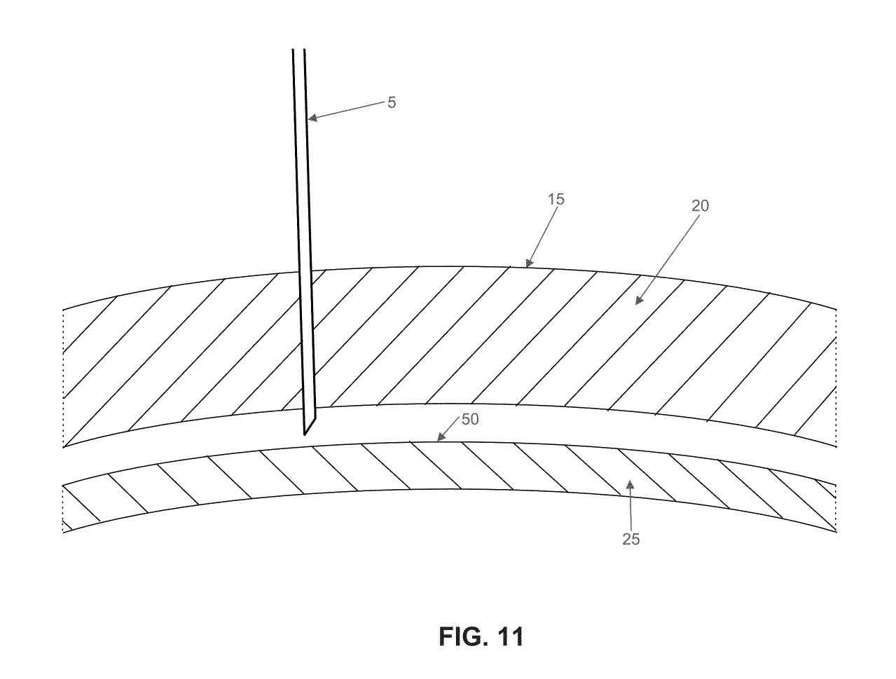

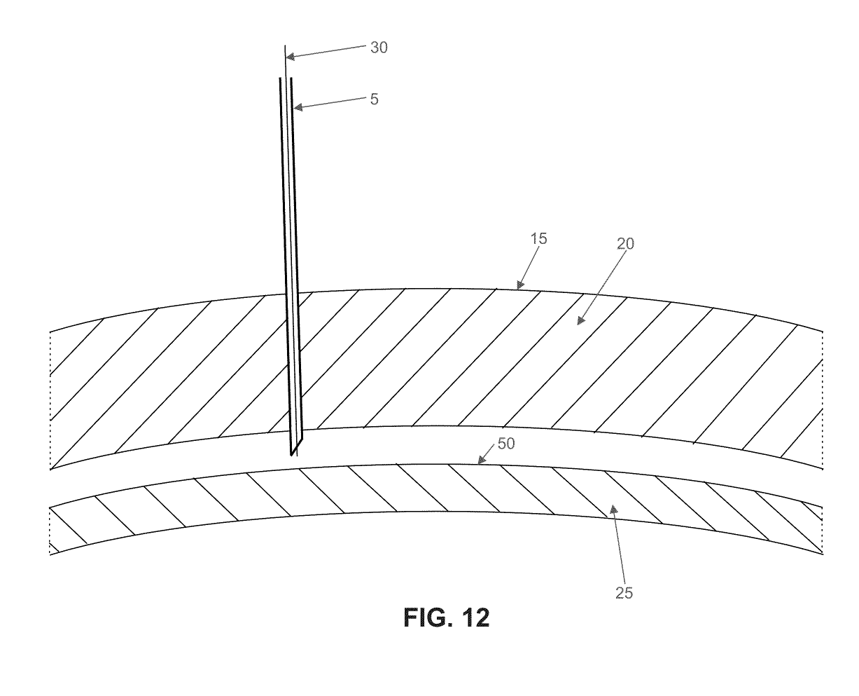

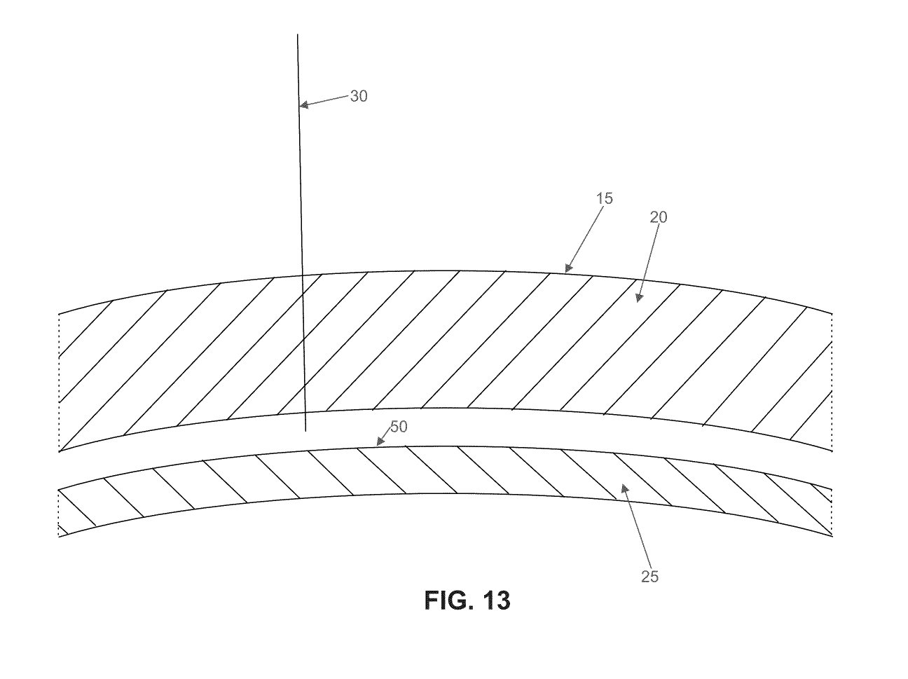

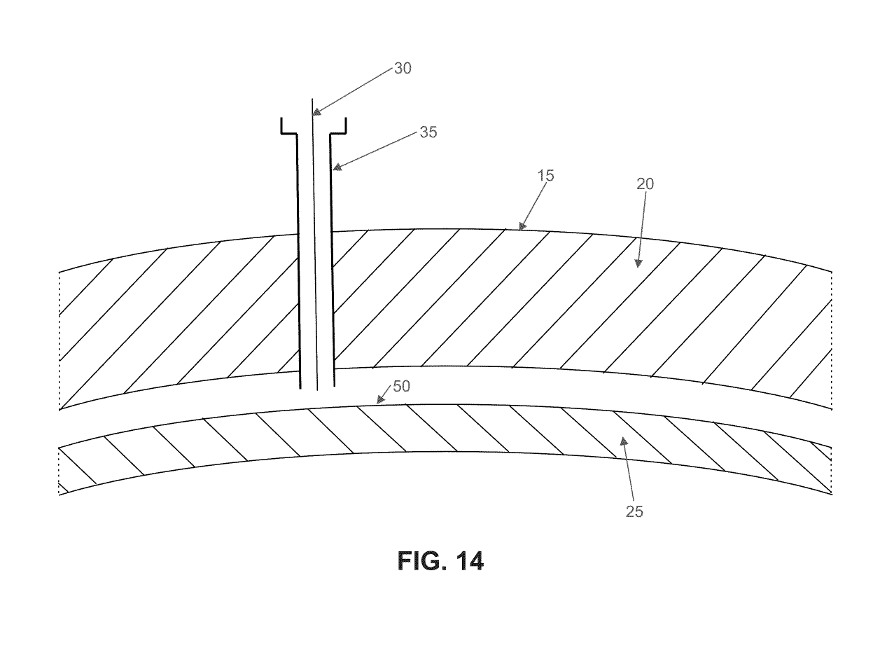

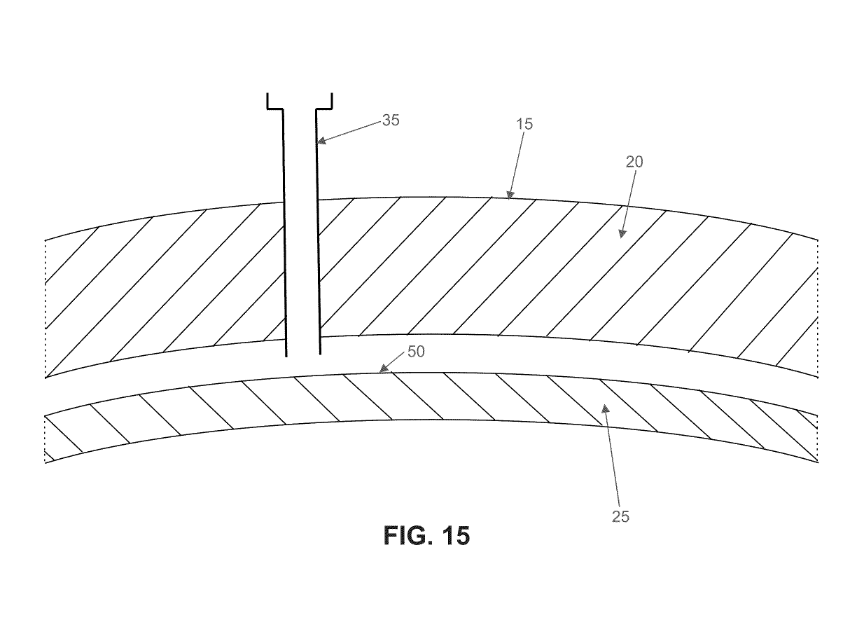

In one preferred form of the present invention, the new approach for arthroscopically accessing the interior of a hip joint comprises the following steps. First, the patient's leg is placed under traction so as to dislocate the femoral head from the acetabular socket. This action creates a gap, or opening, between the femoral head and the acetabular socket, thereby allowing the interior surfaces of the joint to be accessed. Under fluoroscopic guidance, and looking now at FIG. 10, the surgeon then advances needle 5 (with stylet 10) down to, but not through, the outer surface 50 of capsule 25 (FIG. 10). The extent of needle advancement can be determined by fluoroscopy or “surgeon feel”, or both. Next, stylet 10 is removed from needle 5 (FIG. 11). Then guidewire 30 is advanced through needle 5 so that the guidewire extends down to, but not through, the outer surface 50 of capsule 25 (FIG. 12). Next needle 5 is removed, leaving guidewire 30 extending from the surface of the skin 15 down to, but not through, the capsule (FIG. 13). Then a skin incision may or may not be made about guidewire 30. Next, an access cannula 35 is advanced over guidewire 30 and down to, but not through, capsule 25 (FIG. 14). Guidewire 30 is then removed (FIG. 15).

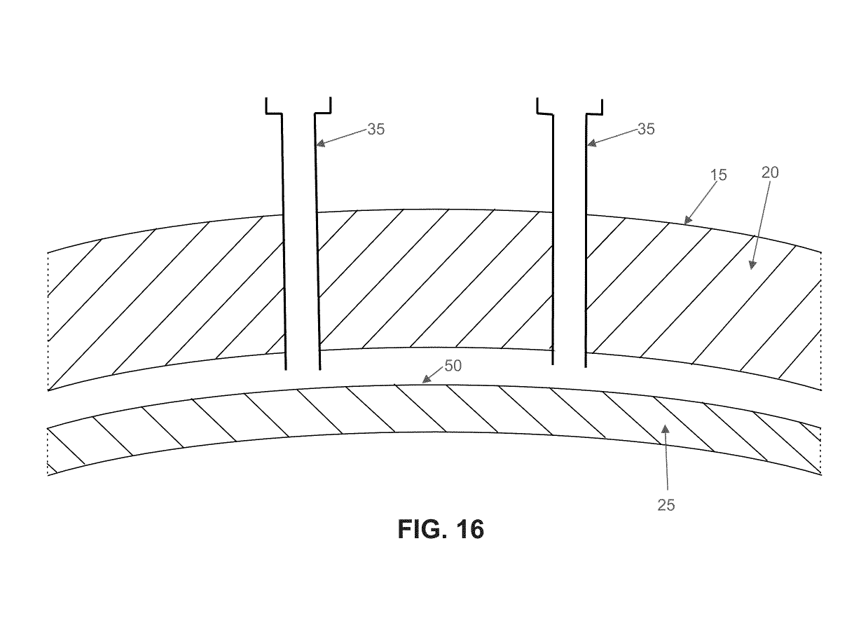

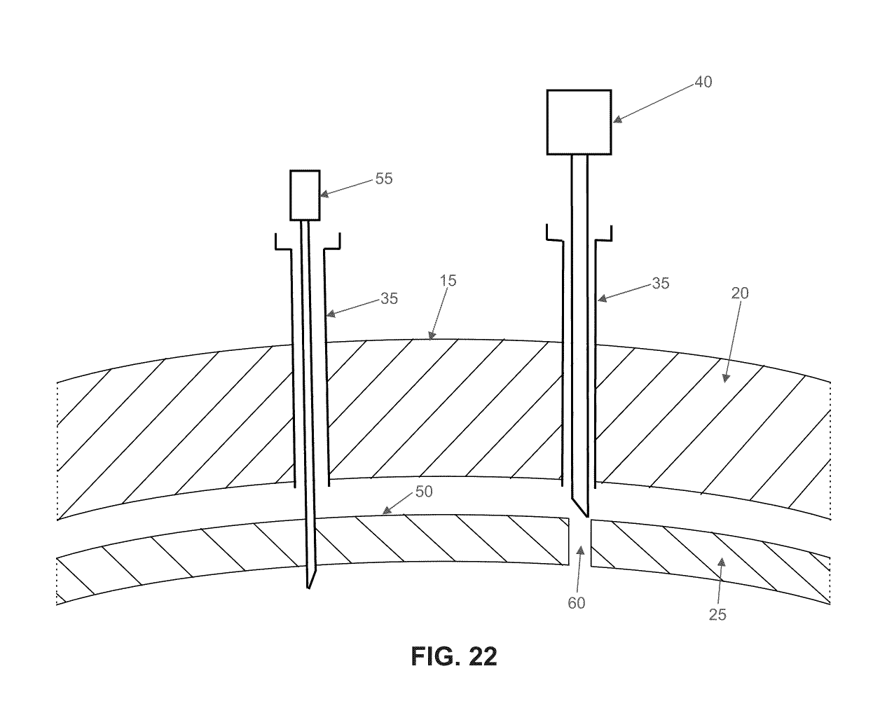

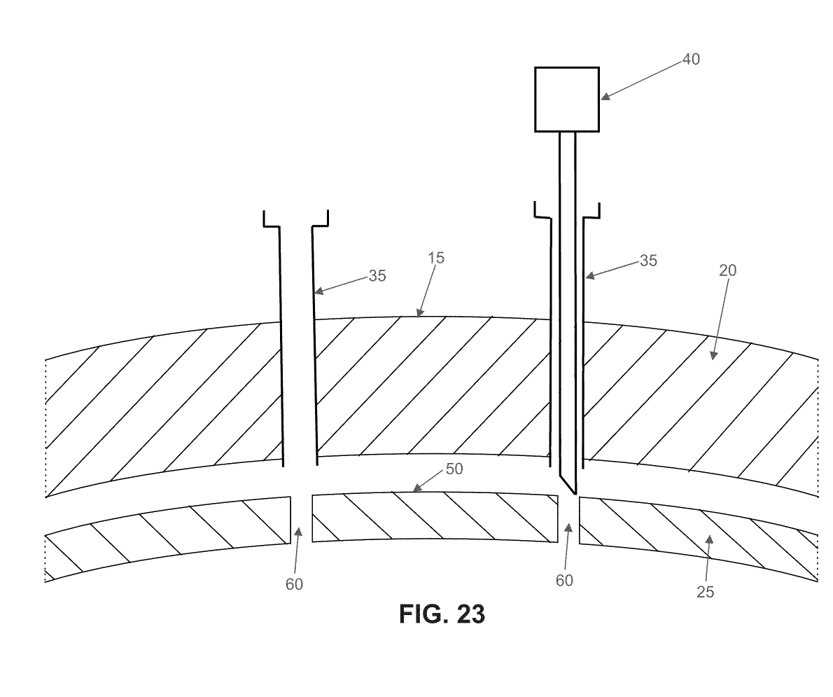

At this point at least one additional access cannula is introduced into the tissue using the same technique (FIG. 16). Preferably at least two additional access cannulas are introduced into the tissue using the same technique, however, only one additional access cannula is shown in FIGS. 16-29 for the sake of clarity. Again, during deployment of the additional access cannulas into the tissue, care is taken to prevent needle 5, guidewire 30 and/or access cannulas 35 from penetrating the capsule.

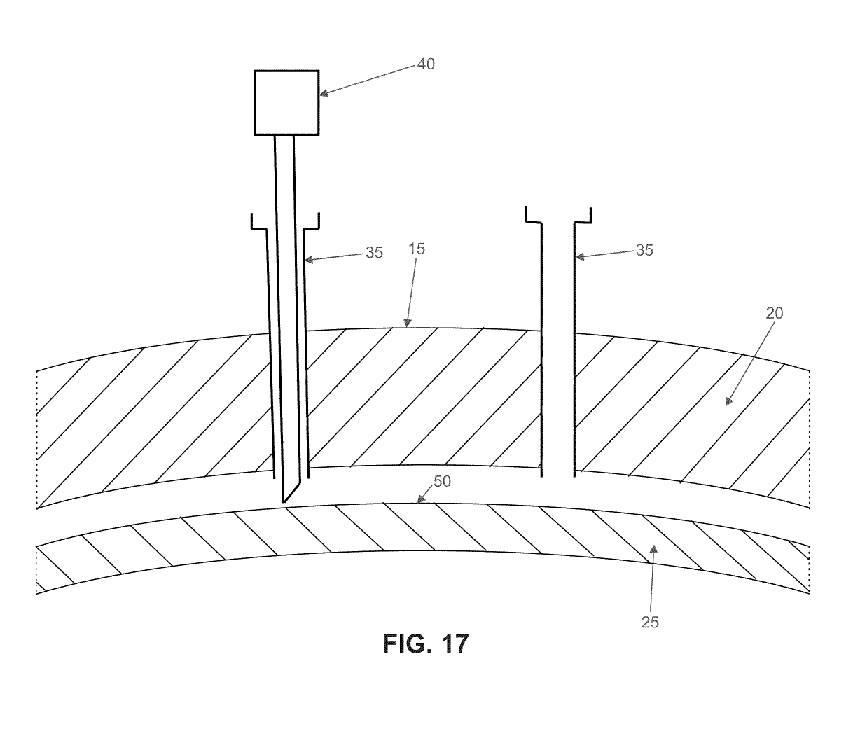

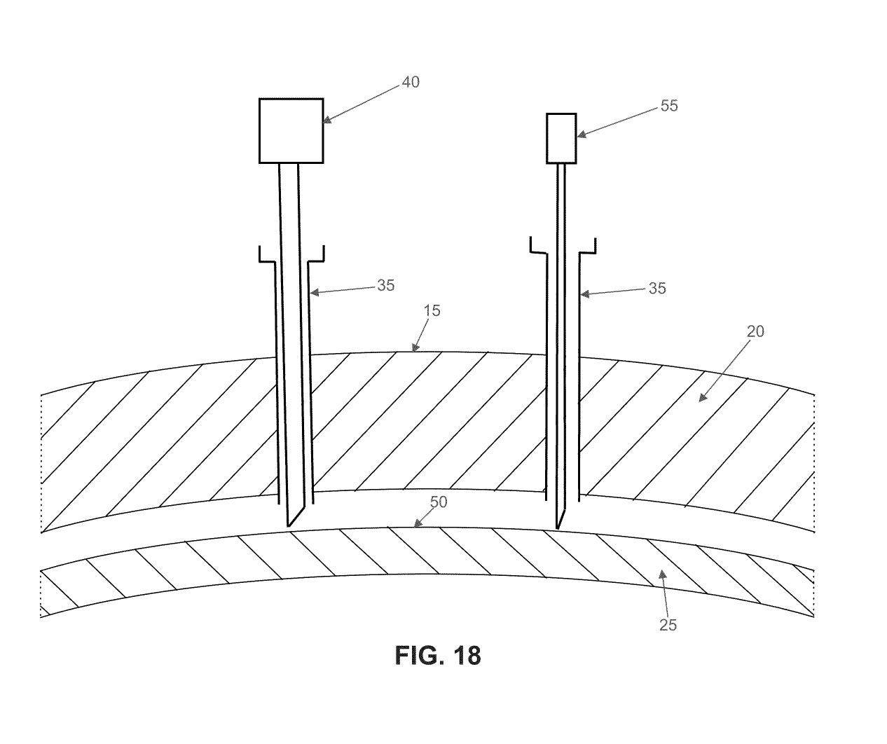

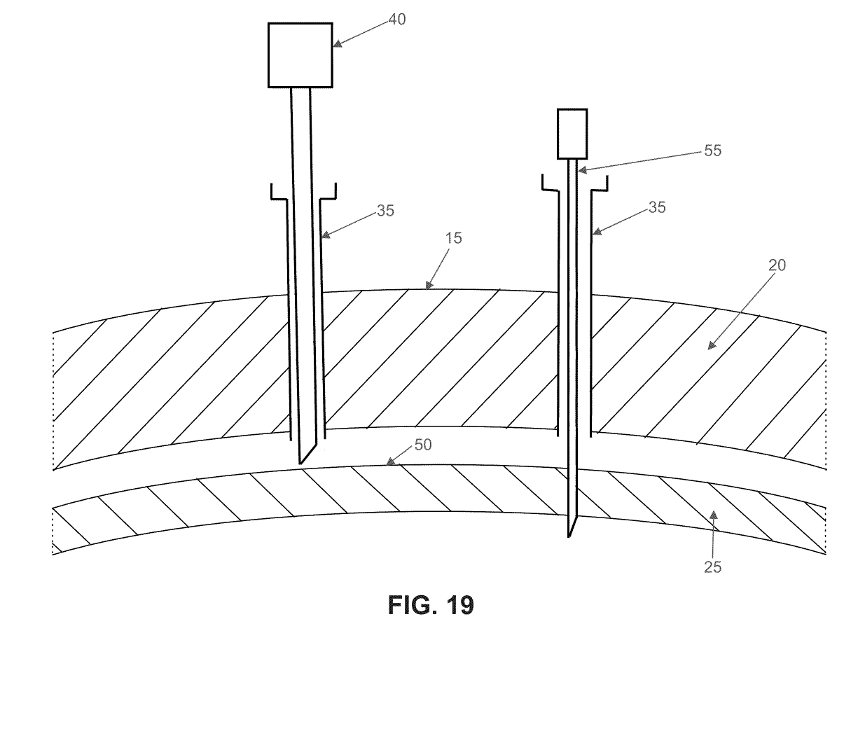

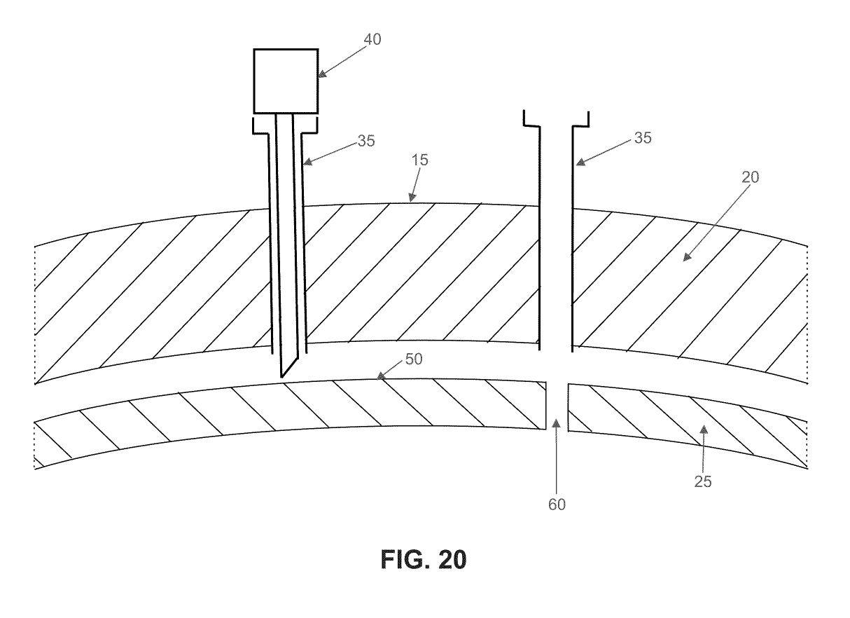

Next, an arthroscope 40 is advanced through one of the access cannulas 35 so that the outer surface of the capsule can be visualized (FIG. 17). Then, while the outer surface of the capsule is being visualized by arthroscope 40, a cutting instrument 55 (e.g., an arthroscopic scalpel) is advanced through the other access cannula 35 (FIG. 18). While under such visualization, cutting instrument 55 is then used to make a cut, or opening, 60 through capsule 25 (FIGS. 19 and 20). The surgeon is able to make this cut or opening 60 with precision, and without fear of unintentionally plunging into the underlying anatomical structure of the joint, due to (i) the nature of the cutting instrument (i.e., it is a cutting scalpel, not a needle), (ii) the controlled application of the cutting instrument to the tissue (i.e., it is applied directly against the capsule before any cutting occurs, and is not driven through numerous layers of intervening tissue before it encounters the capsule), and (iii) the direct visualization of the capsule penetration which is provided by the arthroscope (i.e., the cut is made into the capsule while under direct visualization, it is not made “blind” as is the case with the prior art). Cut 60 is preferably made in the region of the capsule that resides over the gap between the acetabular rim and femoral head. This cut provides full access into the interior of the hip joint.

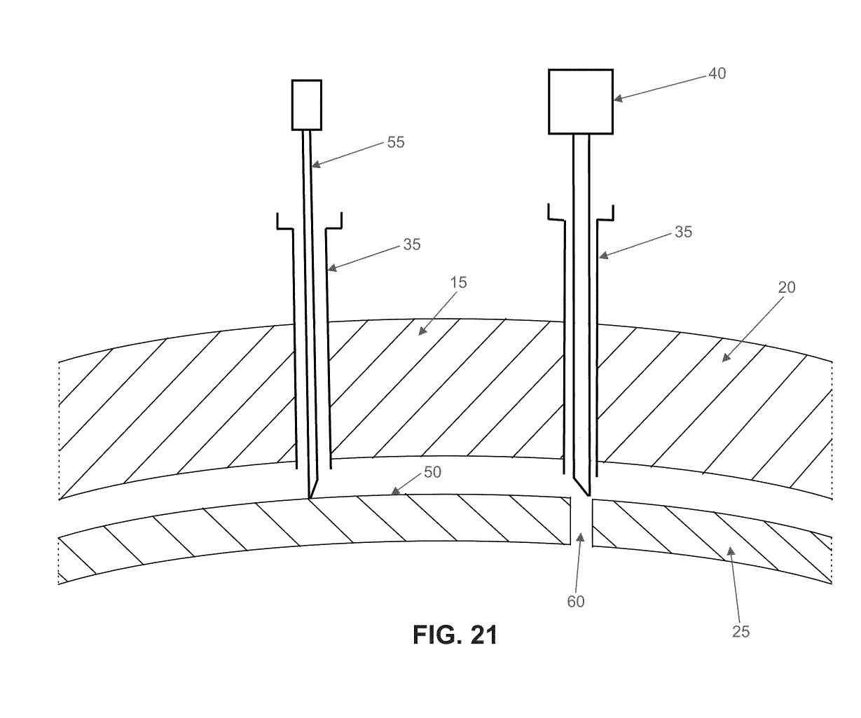

The same process is then repeated so as to create a cut in the capsule below each of the access cannulas positioned in the patient (FIGS. 21-23).

It should be appreciated that, when making cuts 60, the surgeon may use anatomical landmarks to identify the location of a cut. In one embodiment, the anatomical landmark is the direct head or indirect head of the rectus femoris. In one embodiment, the cut is made between the lateral and medial arms of the iliofemoral ligament.

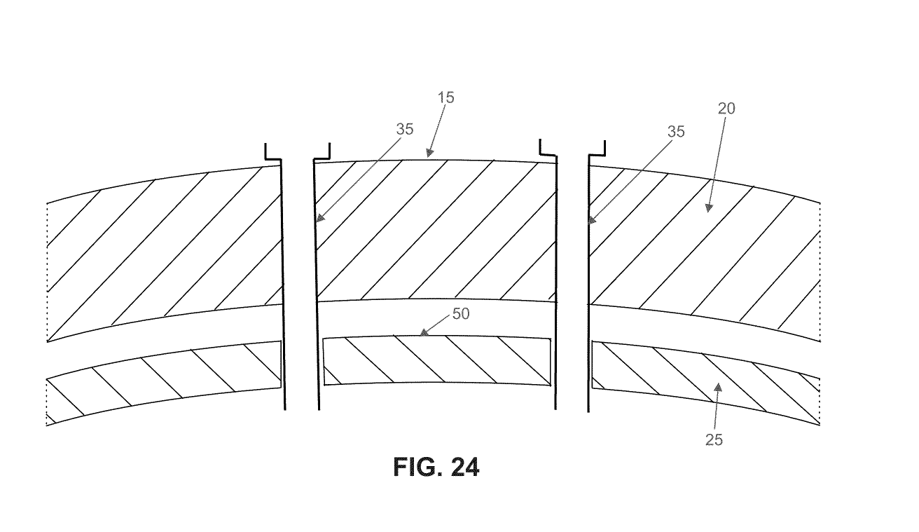

Once these cuts (or openings) have been created in the capsule beneath each of the access cannulas, the access cannulas 35 are then advanced through the cuts made in the capsule and into the interior of the hip joint, whereby to provide a corridor from the surface of the skin down into the interior of the joint (FIG. 24).

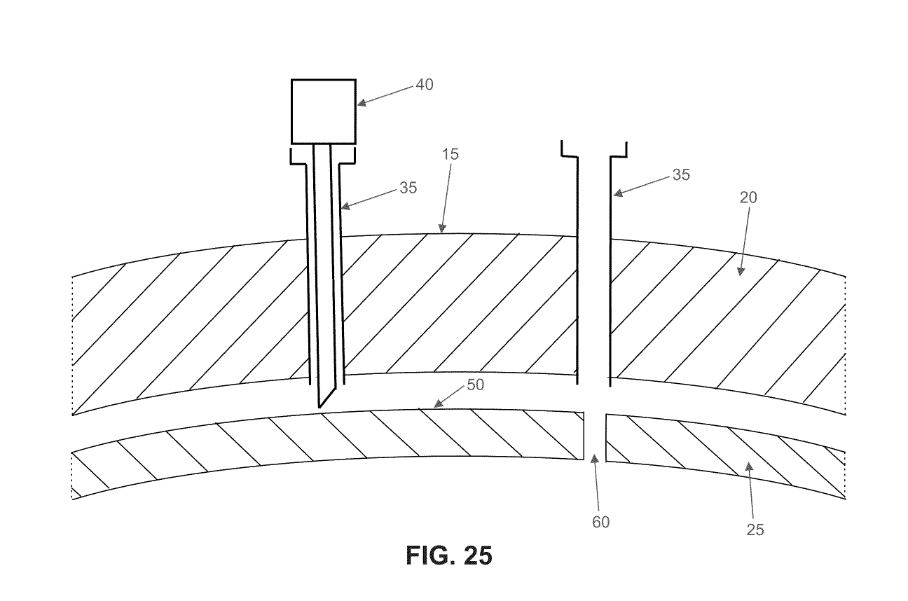

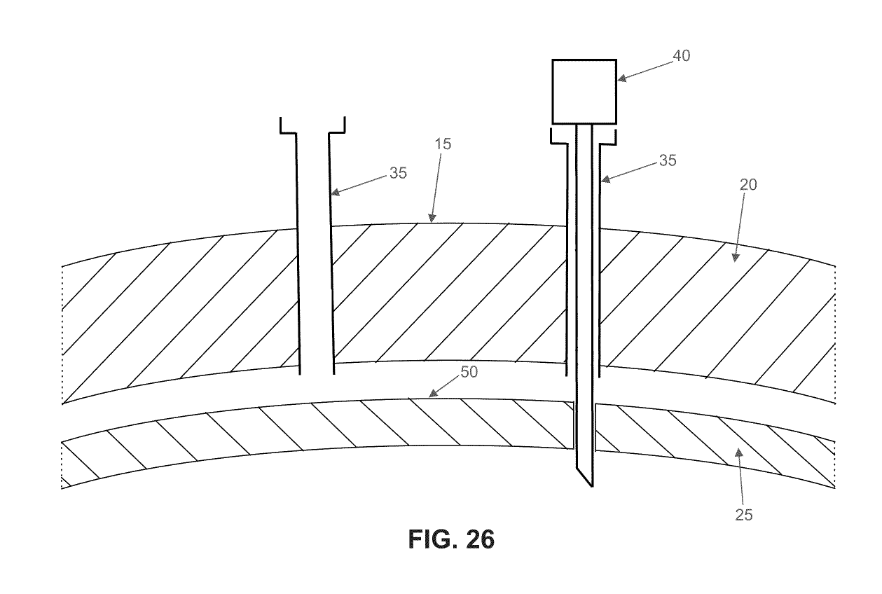

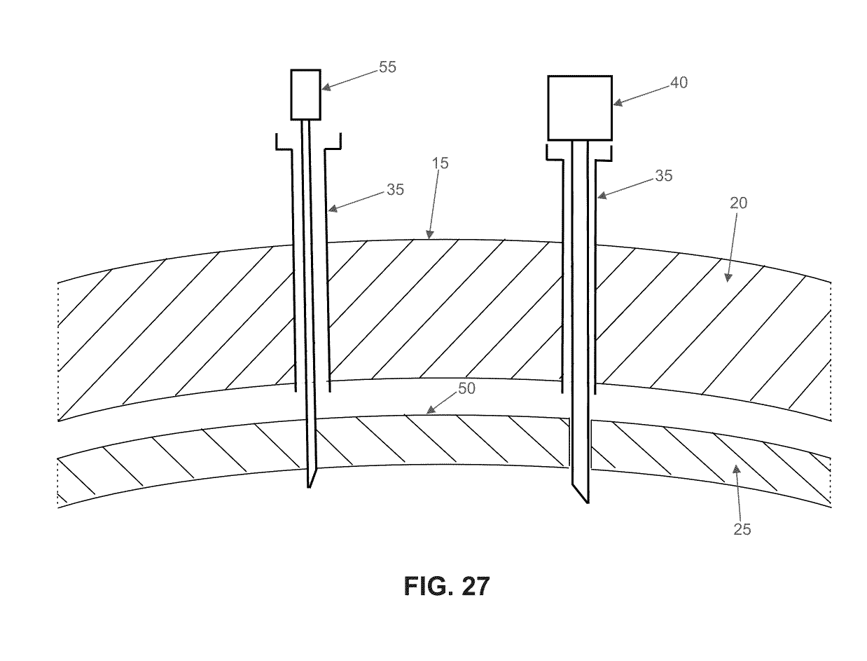

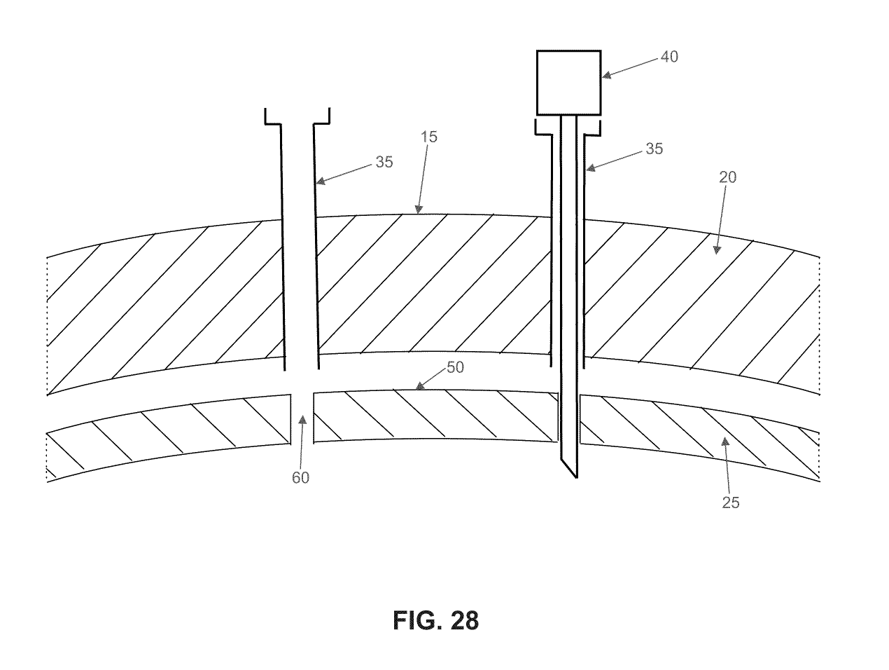

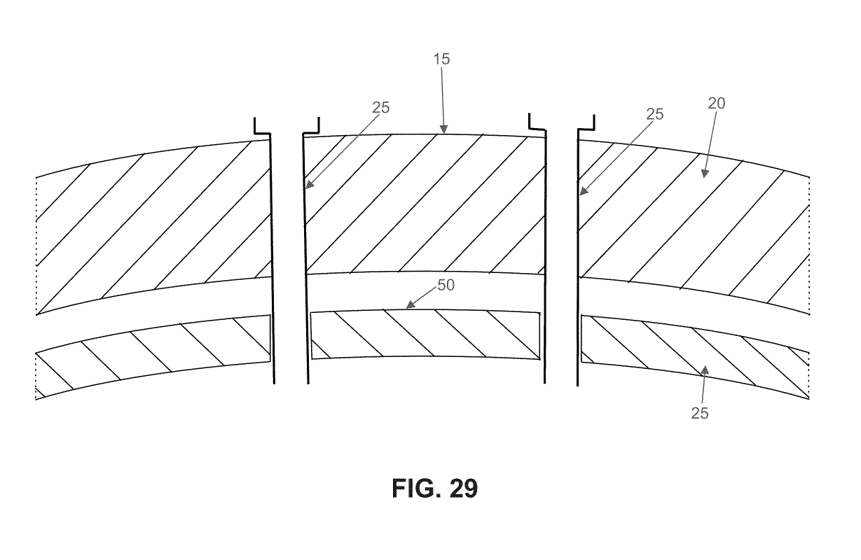

Alternatively, if desired, once a first cut 60 has been made in the capsule (FIG. 25), the arthroscope can be advanced through that cut (FIG. 26) so as to visualize the underside of the capsule. While the underside of the capsule is so visualized, additional cuts are made in the capsule (FIGS. 27 and 28). Once all of the cuts 60 have been made in the capsule, access cannulas 25 are then advanced through the cuts made in the capsule and into the interior of the joint (FIG. 29).

In one preferred form of the invention, the access cannulas 25 may comprise telescoping access cannulas of the sort taught in pending prior U.S. patent application Ser. No. 12/631,514, filed Dec. 4, 2009 by James Flom et al. for METHOD AND APPARATUS FOR ACCESSING THE INTERIOR OF A HIP JOINT, INCLUDING THE PROVISION AND USE OF A NOVEL TELESCOPING ACCESS CANNULA AND A NOVEL TELESCOPING OBTURATOR (Attorney's Docket No. FIAN-3143), which patent application is incorporated herein by reference. These telescoping access cannulas are designed to allow their overall length to be adjusted in situ, which can be highly advantageous when the distal tip of the access cannula is to be advanced from a position outside of the capsule to a position inside of the capsule.

It should be appreciated that variations may be made to the approach described above without departing from the scope of the present invention.

For example, the surgeon may not place an access cannula in the patient, but rather introduce an arthroscopic instrument through the anatomical tissue pathway created by the needle.

Additionally, the capsule may not initially be cut at the gap between the acetabular rim and femoral head—in an alternative approach, the cut may be made in the region of the capsule that is over the femoral neck. Accessing the joint over the femoral neck may be safer then accessing the joint over the gap between the femoral head and the acetabular cup, as there may be less likelihood to damage cartilage or soft tissue structures during capsule penetration. In this alternative embodiment, the cut could subsequently be extended from the femoral neck to the gap between the acetabular rim and femoral head, thus gaining access to the hip interior.

In yet another alternative embodiment, a balloon or other space-creating structure may be disposed between outer tissue 20 (skin, muscle, etc.) and capsule 25 prior to advancing needle 5 (with stylet 10) through outer tissue 20 and down to, but not through, the capsule. Such an approach can make it easier to appropriately position needle 5, guidewire 30, access cannulas 35, arthroscope 40 and/or cutting instrument 55 in the gap between tissue 20 and capsule 25.

Furthermore, once passageways have been created through capsule 25 (e.g., the placement of access cannulas through capsule 25), one or more balloons can be placed within the central compartment (i.e., the gap between the head of the femur and the acetabular cup) so as to further distract and/or otherwise support the joint. Furthermore, one or more balloons may be placed in the peripheral compartment (i.e., the space between the capsule 25 and the neck of the femur) so as to lift the capsule away from the femur and/or provide a fulcrum structure for levering the femur relative to the acetabular cup. These and other balloon applications are disclosed in pending prior U.S. patent application Ser. No. 12/726,268, filed Mar. 17, 2010 by Julian Nikolchev et al. for METHOD AND APPARATUS FOR DISTRACTING A JOINT, INCLUDING THE PROVISION AND USE OF A NOVEL JOINT-SPACING BALLOON CATHETER AND A NOVEL INFLATABLE PERINEAL POST (Attorney's Docket No. FIAN-28424953), which patent application is incorporated herein by reference.

It should also be appreciated that the cutting instrument could have various embodiments. It could be a mechanical blade, a radio-frequency device, an ultrasonic cutter, an oscillating blade, or any other instrument consistent with the present invention and capable of cutting tissue. The cutting instrument may be used over a guidewire or a switching stick.

### USE OF THE PRESENT INVENTION FOR OTHER APPLICATIONS

It should be appreciated that the present invention may be used for accessing joints other than the hip joint, e.g., it may be used to access the shoulder joint.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for arthroscopically accessing a region of a joint, wherein the joint has a capsule disposed intermediate at least one layer of outer tissue and the joint, the method comprising:
arthroscopically positioning visualization apparatus adjacent to an exterior surface of the capsule; and
while visualizing the exterior surface of the capsule, arthroscopically forming an opening through the capsule.

2. A method according to claim 1 wherein the visualization apparatus is arthroscopically positioned adjacent to an exterior surface of the capsule by advancing the visualization apparatus through a first access cannula which extends through the at least one layer of outer tissue.

3. A method according to claim 2 wherein the opening through the capsule is formed using a cutting tool, and further wherein the cutting tool is advanced to the capsule through a second access cannula extending through the at least one layer of outer tissue.

4. A method according to claim 2 wherein the first access cannula is advanced through the at least one layer of outer tissue by advancing the first access cannula along a guidewire which extends through the at least one layer of outer tissue.

5. A method according to claim 4 wherein the guidewire is advanced through the at least one layer of outer tissue by passing a needle through the at least one layer of outer tissue, advancing the guidewire through the needle, and then removing the needle.

6. A method according to claim 3 wherein the second access cannula is advanced through the at least one layer of outer tissue by advancing the second access cannula along a guidewire which extends through the at least one layer of outer tissue.

7. A method according to claim 6 wherein the guidewire is advanced through the at least one layer of outer tissue by passing a needle through the at least one layer of outer tissue, advancing the guidewire through the needle, and then removing the needle.

8. A method according to claim 1 wherein the location of the opening through the capsule is determined by visualizing at least one anatomical landmark with the visualization apparatus.

9. A method according to claim 1 wherein the location of the opening through the capsule is adjacent to the interior of the joint.

10. A method according to claim 1 wherein the location of the opening through the capsule is adjacent to an exterior surface of a constituent bone of the joint.

11. A method according to claim 1 wherein the visualization apparatus comprises an arthroscope.

14. A method according to claim 3 wherein the cutting tool comprises an arthroscopic scalpel.

15. A method according to claim 1 wherein the joint comprises the hip joint.

16. A method according to claim 1 comprising the additional step of forming a second opening through the capsule.

17. A method according to claim 16 wherein the second opening is formed through the capsule while the location of the second opening is visualized using the visualization apparatus.

18. A method according the claim 17 wherein the visualization apparatus is aligned with the first opening while the second opening is being formed through the capsule.

19. A method according to claim 18 wherein the visualization apparatus is arranged to visualize the exterior surface of the capsule while the second opening is being formed through the capsule.

20. A method according to claim 18 wherein the visualization apparatus is arranged to visualize an interior surface of the capsule while the second opening is being formed through the capsule.

21. A method according to claim 2 comprising the further step of advancing the first access cannula through the opening.

22. A method according to claim 21 wherein the first access cannula comprises a telescoping access cannula.

23. A method according to claim 1 comprising the further step of advancing a balloon through the opening.

24. A method according to claim 1 wherein a balloon is inflated adjacent to the exterior surface of the capsule before forming the opening.
